(19) United States
(12) Patent Application Publication
(10) Pub. No.: US 2010/0029546 A1
(43) Pub. Date: Feb. 4, 2010

(54) preparation method of galactosyl-has magnetic nanoparticles containing adriamycin

(76) Inventor: Yangde Zhang, Hunam (CN)

Correspondence Address: KNOBBE MARTENS OLSON & BEAR LLP, 2040 MAIN STREET, FOURTEENTH FLOOR, IRVINE, CA, 92614, US

(21) Appl. No.: 11/663,201

(22) PCT Filed: Sep. 24, 2004
(86) PCT No.: PCT/CN04/01091

§ 371 Date: Jun. 8, 2009

(30) Foreign Application Priority Data
Aug. 11, 2004 (CN) 200410046650.6

(51) Int. Cl. A61K 38/38; C07K 1/107; A61P 35/04

(52) U.S. Cl. 514/6; 530/369

(57) ABSTRACT

Preparation of galactose albumin adriamycin magnetic nanoparticles: in order to prepare galactose albumin magnetic nanoparticle, cottonseed oil and magnetic nano powder is needed. Mix galactose albumin, adriamycin and magnetic nanoparticle at a proportion, and get the particle through emulsification in cottonseed oil, heating for solidification, and diethyl ether washing. This invention couples galactose to the surface of nanoparticle to form galactose nanoparticle, which targets actively and passively to improve the drug targeting level to liver. Modifying albumin adriamycin magnetic nanoparticles with galactose enhances its targeting level.

### BACKGROUND OF THE INVENTION

1. Field of the Invention

The present invention is related to a method for the preparation of nanoparticle loaded genetic medicine treating hepatocarcinoma.

2. Description of the Related Art

Primary carcinoma of liver is one of the most epidemic malignant tumors. At present, the conventional therapies include surgical operation and chemical treatment and so on, and the complete resection is quite few, and the targeting property of chemical treatment is poor. Although genetic therapy has obtained a lot of achievement for recent years, the results of clinical experiments are not satisfied. For the improvement of diagnosis of early hepatocarcinoma, the surgical complete resection has improved greatly, but it is easy to recur. The main reason of the recidivation is the remained micro cancer tissues after resection. The chemical treatment for the micro cancer tissues is not only hard to get a satisfied curative effect, but also cannot get rid of the toxicity to healthy cells.

### SUMMARY OF THE INVENTION

The goal of this invention is to provide a method to prepare ADM-GHMN, a kind of genetic medicine for treating hepatocarcinoma, which has good targeting property and low toxicity.

The technical project of the invention: To prepare galactosyl-HAS at first, mix up adriamycin, galactosyl-HAS, magnetic powder according to a certain proportion, through ultrasonic emulsification, heating-dystrophy-congelation, and washing with ether, the mixture can be turned into nanoparticles loaded drug.

The invention is to combine galactosyl with the surface of nanoparticle loaded drug to prepare galactosyl-nanoparticles, which has initiative targeting property and passive targeting property, so it realizes better liver targeting property. The decoration HAS-magnetic-adriamycin-nanoparticles with galactosyl strengthen the targeting property of HAS-magnetic-adriamycin-nanoparticles.

The detail of the preparation in the invention is as following.

To prepare a sample of ADM-GHMN, cotton seed oil and magnetic-nanoparticles were needed at first.

The refine of cotton seed oil: To heat common cotton seed oil to 30˜50° C. at least, adding into NaOH during stirring to saponify free fat completely, then the solution was heated to 60˜70° C. and maintain the level for 30 min to make full saponification, then get the oil solution after filtration. The oil solution was heated to 50° C., and adding certain amount of active carbon. It was heated to 80° C. and maintain the level for 0.5 hour; the decolorizer should be filtrated at the high temperature; the remained oil solution was added in dehydrating CaCl2. After one night, the solution was filtrated to get the needed cotton seed oil.

The preparation of magnetic-nanoparticles: To get FeCl₃.6H₂O weight 0.85 g (3.1 mol), FeCl₂.4H₂O weight 0.3 g (1.5 mol), and they were solved in 200 ml solution with 0.1% Tween-80 in it. 1.5 mol/l NH₄OH was added slowly in the solution to pH=8 to hydrolyze fully. To separate the Fe₃O₄ crystal in the solution used magnet. It had been washed for 3 times with distilled-water, then disperse in 20 ml distilled-water.

The preparation of ADM-GHMN: The prepared magnetic powder was dealt with ultrasound for 2 minutes, and get 900 mg of it, 200 mg galactosyl-HAS, which were dissolved in 0.5 ml distilled-water. Resolved 10 mg adriamycin in 0.5 ml distilled-water and mixed up symmetrically, then 15 ml refined cotton seed oil is added into it. The solution had been emulsified for 10 minutes with ultrasound at 4° C. After symmetrical disperse, the solution was added into 120° C. 50 ml refined cotton seed oil according to the speed of 100 drops per-minute at the stirring speed of 2000 r/min, and maintain the reaction for 20 minutes, then cool it quickly to room temperature, and add 30 ml aether in it; the solution had been centrifuged for 15 minutes at 3000 r/min with centrifugal separator; the upper layer solution was got rid of; the leftover had been washed for 4 times, then was dried at 4° C. At last, ADM-GHMN in the invention was gained.

### EXPERIMENTAL DATA

(1) The magnetism test of ADM-GHMN. To drop one drop of the solution on slide glass, the particles movement could not be observed under microscope without any magnetic field. The particles movement could not be observed after 3 minutes too. With a magnet having magnetic intensity of 4250 Gauss aside for 30 seconds, it could be observed that most of the nanoparticles moved towards the magnet, and they gathered at the side of the magnet finally. If the magnet was taken away, the nanoparticles moved more slowly, and they were arranged along the magnetic line of force.
(2) To chose the best method to prepare ADM-GHMN with orthogonal design. Taking solidification time, galactose density, solidification temperature and stirring speed as technical parameters to observe their infection to the particle size, drug load and trap efficiency.
(3) To chose the time of ultrasound, galactose density, solidification temperature and stirring speed as factors, and taking 4 levels in each of them according to the references. The distribution proportion, drug load, trap efficiency of the nanoparticles within 120˜300 nm were taken as the indexes to observe the preparation technology. (Tab 1) A final orthogonal index was gained according to the distribution proportion, drug load, trap efficiency of the nanoparticles within 120˜300 nm. The formula is following:

$$Di = \frac{( Yi - Ymix )}{( Y max - Ymix )}$$

$$DF = [ {\prod }_{i = 1}^{3} Di ] 1 / 3$$

Yi is experimental data; Ymax, Ymin are the maximum data and the minimum data could be chosen according to the result of former experiment and experience. Di is a single orthogonal index, when Yi<Ymin, then Di=0, when Yi>Ymax, then Di=1.DF is the final orthogonal index based on single the orthogonal indexes. The Yi got from experiments and the Ymax, Ymin set already together can calculate for Di, then get DF. The maximum values and the minimum values are showed here (Tab A). To measure every orthogonal index, and calculate final orthogonal index according to the orthogonal design table L16(45). The result of analysis of variance with drug load, particle size and trap efficiency are showed (Tab 2, 3, 4). The orthogonal term is A1B4C4D4. There is no obvious difference among the infection of every galactose density to final orthogonal index (P>0.05), but the drug load declined with the rise of galactose density in Tab 3 (P<0.05). It was in accordance with the references, and the galactose density of galactosyl-HAS was chosen at 23-30 according to the drug load and saturated degree of the receptors in liver. The detail is as following: galactosyl-HAS with 23-30 galactose density weight 200 mg, magnetic powder weight 900 mg and adriamycin weight 10 mg were mixed up symmetrically, then 15 ml refined cotton seed oil was added in, and dealt with ultrasound for 10 minutes at 4° C. The solution was added in 50 ml cotton seed oil heated first to 120° C. at the speed of 100 drops per-minute with stirring (2000 r/min). The reaction would maintain for 20 minutes, then it was cooled down to room temperature, and washed by 30 ml aether. The solution was centrifuged for 15 min at 3000 r/min; the upper layer of the solution was removed; the leftover was washed with aether 4 times; the nanoparticles were prepared, and could be stored at 4° C. The average particle size is 197±32 nm (FIG. 3.1˜3.3). The SEM picture is showed in (FIG. 4.1˜4.2); the drug load is 48.79±4.47 μg/mg; the trap efficiency is 94.34±3.32%. It indicates that the nanoparticles prepared with the orthogonal term has fine size, high drug load and trap efficiency, and it can achieve the standard of the drug used through vien. The orthogonal term is stable, and the experiment has fine repeatability.

TABLE A
Maximum Values and the Minimum Values

| Index | Ymax | Ymin |
|---|---|---|
| Average Size (%) | 100 | 50 |
| Drug Loading (ug/mg) | 60 | 30 |
| Trap Efficiency (%) | 100 | 60 |

Phenol-sulphuric acid method measures the galactose in galactosyl-HAS-nanoparticles. The galactose in the nanoparticles measured is 58.75 ± 3.53 μg/mg.

Recovery rate experiment. The average recovery rate is 99.74±1.35%.

Drug releases in vitro. According to dynamic dialysis, taking physiological saline as medium, 4 ml of the solution would be taken at certain time, and the same volume of the medium would be added in. The absorbency was measured to get the solving efficiency according to the standard curve formula. At the same time, the drug releasing experiment of adriamycin standard substance was done in order to observe the control release of the dialysis bag. The release of adriamycin standard substance has achieved 91.34%, and the drug almost has released completely; The drug in the nanoparticles releases quickly at early stage, then it releases slowly, the releasing efficiency being 59.73% (Tab 6). It indicated that the nanoparticles could release drug slowly.

TABLE 1
L₁₆ (4⁵) Orthogonal Design

| | Factor | | | | | |
|---|---|---|---|---|---|---|
| Number | 1 (A) | 2 (B) | 3 (C) | 4 (D) | 5 | DF |
| 1 | 1 | 1 | 1 | 1 | 1 | 0.1709 |
| 2 | 1 | 2 | 2 | 2 | 2 | 0.6627 |
| 3 | 1 | 3 | 3 | 3 | 3 | 0.8363 |
| 4 | 1 | 4 | 4 | 4 | 4 | 0.9224 |
| 5 | 2 | 1 | 2 | 3 | 4 | 0.3743 |
| 6 | 2 | 2 | 1 | 4 | 3 | 0.6371 |
| 7 | 2 | 3 | 4 | 1 | 2 | 0.6527 |
| 8 | 2 | 4 | 3 | 2 | 1 | 0.7920 |
| 9 | 3 | 1 | 3 | 4 | 2 | 0.5223 |
| 10 | 3 | 2 | 4 | 3 | 1 | 0.6942 |
| 11 | 3 | 3 | 1 | 2 | 4 | 0.6145 |
| 12 | 3 | 4 | 2 | 1 | 3 | 0.6415 |
| 13 | 4 | 1 | 4 | 2 | 3 | 0.4973 |
| 14 | 4 | 2 | 3 | 1 | 4 | 0.4702 |
| 15 | 4 | 3 | 2 | 4 | 1 | 0.6840 |
| 16 | 4 | 4 | 1 | 3 | 2 | 0.6572 |
| AV 1 | 0.648 | 0.391 | 0.520 | 0.484 | | |
| AV 2 | 0.614 | 0.616 | 0.591 | 0.642 | | |
| AV 3 | 0.618 | 0.697 | 0.655 | 0.641 | | |
| AV 4 | 0.577 | 0.753 | 0.692 | 0.691 | | |

| | Type III | | | | |
|---|---|---|---|---|---|
| Source | Sum of Squares | df | Mean Square | F | Sig. |
| Galactose Density | 1.009E−02 | 3 | 3.363E−03 | .951 | .516 |
| Mix Speed | .302 | 3 | .101 | 28.438 | .011 |
| Solidification Time | 6.885E−02 | 3 | 2.295E−02 | 6.492 | .079 |
| Solidification | 9.958E−02 | 3 | 3.319E−02 | 9.389 | .049 |
| temperature | | | | | |
| Error | 1.061E−02 | 3 | 3.535E−03 | | |

TABLE 2
ANOVA of Drug Loading

| | Type III | | | | |
|---|---|---|---|---|---|
| Source | Sum of Squares | df | Mean Square | F | Sig. |
| Galactose Density | 235.985 | 3 | 78.662 | 1218.497 | .000 |
| Mix Speed | .590 | 3 | .197 | 3.049 | .192 |
| Solidification | 5.711 | 3 | 1.904 | 29.490 | .010 |
| Time | | | | | |
| Solidification | 15.032 | 3 | 5.011 | 77.616 | .002 |
| Temperature | | | | | |
| Error | .194 | 3 | 6.456E−02 | | |
| Total | 39407.081 | 16 | | | |

TABLE 3
ANOVA of Average Size

| | Type III | | | | |
|---|---|---|---|---|---|
| Source | Sum of Squares | df | Mean Square | F | Sig. |
| Galactose Density | 465.830 | 3 | 155.277 | 7.260 | .069 |
| Mix Speed | 2324.316 | 3 | 774.772 | 36.223 | .007 |
| Solidification Time | 96.530 | 3 | 32.177 | 1.504 | .373 |
| Solidification | 300.072 | 3 | 100.024 | 4.676 | .119 |
| Temperature | | | | | |
| Error | 64.168 | 3 | 21.389 | | |
| Total | 104643.85 | 16 | | | |

TABLE 4
ANOVA of Trap Efficiency

| | Type III | | | | |
|---|---|---|---|---|---|
| Source | Sum of Squares | df | Mean Square | F | Sig. |
| Galactose Density | 10.799 | 3 | 3.600 | 1.183 | .447 |
| Mix Speed | 39.779 | 3 | 13.260 | 4.359 | .129 |
| Solidification Time | 196.939 | 3 | 65.646 | 21.580 | .016 |
| Solidification | 910.054 | 3 | 303.351 | 99.720 | .002 |
| Temperature | | | | | |
| Error | 9.126 | 3 | 3.042 | | |
| Total | 121313.85 | 16 | | | |

*F_{0.05(3,4)=9.28}, F_{0.01(3,4)=29.46}

TABLE 5
Recovery Rate

| Addition of Adriamycin | Measured Value | Recovery Rate |
|---|---|---|
| (ug/ml) | (ug/ml) | (%) |
| 21.65 | 21.34 | 98.57 |
| 21.65 | 21.45 | 99.08 |
| 10.83 | 11.03 | 101.85 |
| 10.83 | 10.68 | 98.61 |
| 6.50 | 6.59 | 101.38 |
| 6.50 | 6.43 | 98.92 |

(n = 5)

TABLE 6
Releasing of adriamycin and drug ADM-GHMN

| | Time (h) | | | | | | | | | |
|---|---|---|---|---|---|---|---|---|---|---|
| | 0.5 | 1 | 2 | 4 | 6 | 8 | 12 | 18 | 24 | 40 |
| Releasing of | 78.21 | 91.34 | 100 | | | | | | | |
| adriamycin (%) | | | | | | | | | | |
| Releasing of | 23.22 | 25.08 | 29.87 | 34.64 | 37.53 | 40.64 | 44.80 | 50.77 | 59.73 | 78.40 |
| ADM-GHMN | | | | | | | | | | |
| (%) | | | | | | | | | | |

### 2. In Vitro Tests

### Medicine Influence on HepG2 Cell's Invasive Power of Hepato-Carcinoma Cell:

To utilize RT-PCR method via ultraviolet Jel image analysis system carrying out gray scale analysis. Measuring variance content of cathepsin mRNA in different groups of RPMI-1640 group, adriamycin group, HSA-magnetic-adriamycin-nanoparticles group, galactosyl-HSA-adriamycin-nanoparticles group, galactosyl-HSA-magnetic-adriamycin-nanoparticles group. Our experimental result showing the expression level of cathepsin mRNA of galactosyl-HSA-magnetic-adriamycin-nanoparticles group is lower compared with RPMI-1640 group, adriamycin group, HSA-magnetic-adriamycin-nanoparticles group and galactosyl-HSA-adriamycin-nanoparticles group, which have significant difference. It proves that galactosyl-HSA-magnetic-adriamycin-nanoparticles have conspicuous depressant effect to tumor cell's invasive power in the condition of combination with magnetic field. Moreover, the effect is more stronger than HSA-magnetic-adriamycin-nanoparticles group and galactosyl-HSA-adriamycin-nanoparticles group. Its possible mechanism is: galactosyl-HSA-magnetic-adriamycin-nanoparticles have the specific target tropism. Its lethal effect on the tumor cell is more powerful compared with ordinary adriamycin and other several kinds medicine contains adriamycin-nanoparticles. The magnetic field has the effect in suppressing malignant tumor cell multiplication at the same time changes the function of the tumor cell's biomembrane, strengthens the cytotoxic effect of the anti-cancer medicine. When the tumor cell was killed massively, Its expression of Cathepsin B-mRNA would inevitably decrease; adriamycin belongs to this type of chemotherapy medicine: Its mechanism depends on adriamycin combining with DNA and inhibit nucleic acid synthesis, It may directly inhibit DNA transcription consequently. Cathepsin B-mRNA's expression would be influenced. This research utilizing nano-medicine targeting to the tumor cell may remarkably enhance adriamycin level in the tumor cell, accordingly reduce expression of Cathepsin B-mRNA; furthermore, it achieved the goal of reduceing the tumor invasive power. Meanwhile, it may affect a series of enzyme's expression related with tumor infiltrate and metastasis through the same functional way, for instance matrix metal protease and so on.

The tumor cell's mobility and the invasion are close to each other. The tumor cell has the ability of amoeba type's movement, and it was confirmed by many people. Simultaneously, many overseas scholars reported that there is a direct ratio relation between the mobility and the invasion ability of the cancer cell. We use the Transwell method to survey invasion result in vitro of tumor cell demonstrating the adriamycin group, HSA-magnetic-adriamycin-nanoparticles group, galactosyl-HSA-adriamycin-nanoparticles group, galactosyl-HSA-magnetic-adriamycin-nanoparticles group and all these four groups can inhibit invasive power of the HepG2 cell. There is significant difference between any two groups. Both HSA-magnetic-adriamycin-nanoparticles group and galactosyl-HSA-adriamycin-nanoparticles group are better in inhibition than adriamycin group (P<0.01). HSA-magnetic-adriamycin-nanoparticles group is different from galactosyl-HSA-adriamycin-nanoparticles group in it. The effect of the latter is stronger than that of the other but there is no significant difference; it is stronger in inhibit invasive power of the HepG2 cell of galactosyl-HSA-adriamycin-nanoparticles group than that of other groups and there is significant difference (P<0.01).

The experiment result proves that several nanoparticle medicine all have obvious inhibition influence to the HepG2 cell and their effects are all stronger than that of adriamycin. In these nanoparticle drugs, we utilize the passive target character and magnetic targeting of nanoparticles and the initiative target character of receptor-mediate developed galactosyl-HSA-adriamycin-nanoparticles and displayed more stronger invasive power to carcinoma cells than that of other experimental medicine.

The mechanism may be the special conduct of galactosyl ligand and the hepatoma carcinoma cell agglutinin's recognition and encytosis in the human being, lead to implement the drug target therapy. Thus, medicine given in the same density could more effectively kill the cancer cell. At the same time, external magnetic field can suppress malignant tumor cell and change the function of biological membrane and the permeability of tumor cell, so that the cytotoxic effect could be strengthened.

The experiment researches of galactose albumin magnetic adriamycin nanoparticles killing hepatocelular cell:

Cell morphologic under optics microscope identified that configuration of the carcinoma cells with galactose albumin magnetic adriamycin nanoparticles combining magnetic field were irregular. The quantity was decreased obviously; cells were rough; profile were strongthened, refracted character were bad; things in cells were confused; a great number of cells were dropped from bottles, which were in karyopyknosis state; the growth condition were worse than others obviously. We found that the chromatin karyopyknosis of apoptosis cells always gathered around nucleus membrane, which assumed moon corpuscle, plasma membrane by condensing or lysing cell plasma encircled cell fragments, there were integrated cell organs in cell plasma. There were karyopyknosis in necrosis cells, but the chromatin distribution were irregular. There were no nucleus fragments appeared; cell plasma swelled obviously; cell organs were always damaged. The experiments results indicated that group of galactose albumin magnetic adriamycin nanoparticles had quadrilateral zone after 24H drug adriamycin experiment; there were no in other groups; there were macromolecules zone in control group after 48H toxicity experiment; there were quadrilateral zone in others; group of galactose albumin magnetical adriamycin nanoparticles were the most obvious. There were all characteristic quadrilateral zone in group of galactose albumin magnetic adriamycin nanoparticles, group of galactose albumin adriamycin nanoparticles, group of albumin magnetical adriamycin nanoparticles, group of adriamycin, which indentificated cell apoptosis induced by galactose albumin magnetical adriamycin-nanoparticles was earlier than others. In 48H drug toxicity experiment, the quadrilateral zone of galactose albumin magnetic adriamycin-nanoparticles group was more obvious than others, which indirectly indentificated cell apoptosis effect induced by galactose albumin magnetic adriamycin-nanoparticles was stronger than others. Applying MTT colorimetric analysis to detect cell activity, and get OD value agter using nanometer drugs containing adriamycin with different concentration, then calculate the suppressing ratio and IC50 under different conditions, which can identify that cell toxicity to tumor cells of galactose albumin magnetic adriamycin-nanoparticles was stronger than others. There were significant difference. (P<0.01), which identified the killing effect on tumor cells of galactose albumin magnetic adriamycin-nanoparticles were obviously stronger than group of galactose albumin magnetic adriamycin-nanoparticles, group of albumin magnetic adriamycin-nanoparticles, and group of adriamycin.

### 3. Animal Experiment:

The target distribution character in mice with planting liver cancer given galactose albumin magnetic adriamycin-nanoparticles via liver artery.

The research of distribution in mice body with planting liver cancer showed: galactose albumin magnetic adriamycin-nanoparticles has obvious liver target character, it is to say, the distribution in liver of galactose albumin magnetic adriamycin-nanoparticles is increased, but the distribution in blood and extrahepatic organs were decreased.

From the table 3-1 it could be found the distribution character of ADM-GHMN+M and MADM-NP+M in rats' vivo. of liver cancer graft. The up take of liver achieves peak after 5 minutes of injection. The former is as 2.69 times as the latter Hepatic extractive ratio debase gradually as time prolonged in all of them. The former hepatic extractive ratio is as 2.3-2.5 times as the latter in every phase for observation. The distribution character of ADM-GHMN+M in blood down, up, then down follow the course of hepatic up take and metabolism answers for characters of distribution and metabolism of target drug. In our trial we found that the radioactivity in tumor tissue is as 7.9 times as normal liver tissue after adding magnetic field to tumor section. In control group, without magnetic field, the radioactivity in tumor tissue is also as 2.7 times as normal liver tissue. It shows that nanoparticles per se have some selectivity without magnetic field. It is familiar with distribution in tumor and non-tumor of other particulates. According report of literature blood vessel density in tumor area is 2-6 times higher than normal liver blood vessel. It is primary reason of distribution of nanoparticles higher in tumor tissue compared to normal liver tissue. It also because powerful licking up activity and great permeability in tumor blood vessel. The collection of magnetic nanoparticles greatly increases in magnetic field.

Under the magnetic field, the distribution of ADM-GHMN+M in transplanting liver cancer rats is similar to the normal rats. The nanoparticles are more concentrated in the tumor tissue and liver, and less in the other organs. The same to the normal rats experiments that we have done before, the radio activity of kidney, heart, lung, small intestinal, spleen, blood and tumor tissue in experimental group is lower than the control group. Therefore, if we want to get the same chemotherapeutics concentration in the tumor area, the drug doses for experimental group will be diminished, so that the chemotherapeutics concentration in normal liver and other normal organs will be decreased obviously. This point will take a great important part in relief of the side effect of chemotherapeutics. According to the distribution of ADM-GHMN+M, we can see that the nanoparticles are more concentrated in liver than other organs.

The result could be found in animal test: the group having NS hepatic artery injection lived 12.7 days on average; the group having lib-adriamycin hepatic artery injection lived 18.7 days on average; HSA-NP hepatic artery injection lived 20.7 days on average; the group having Gal-HAS-NP treatment lived 39.4 days on average. It could be concluded that HSA-NP group, Gal-HSA-NP group, HAS-NP+magnetic field group, and Gal-HSA-NP+magnetic field group had better curative effect (p<0.05) than lib-adriamycin group on liver cancer therapy; Gal-HSA-NP group, HAS-NP+magnetic field group, and ADM-GHMN+magnetic field group had better inhibition effect and higher life extension rates than other groups, and ADM-GHMN+magnetic field group is the highest one; there was no statistical significance between Gal-HSA-NP group and HSA-NP group (p>0.05). From the view of pathological section tumor necrosis extent, Gal-HSA-NP+magnetic field group had most serious necrosis where all of them were badly necrosis and no light necrosis; Gal-HSA-NP group and HAS-NP+magnetic field group had most of medium leveled or badly necrosis and no light necrosis; adriamycin group and HAS-NP group had no badly necrosis but most light or medium leveled necrosis.

It could proved in our previous experiment that ADM-GHMN on the surface of hepatocytes by recipient-ligand function, and the increased magnetic field of tumor enhanced the nanoparticle aggregation which thus increased the concentration of tumor chemotherapeutics. Meanwhile, the aspiration of magnetic nanoparicles produced a longer functional diameter, which caused embolism in tumor blood vessels and lack of blood and oxygen in tumor in order to improve the sensitivity and effect of chemotherapeutics. Without adding magnetic field, the distribution rate of ADM-GHMN in tumorous and normal livers was 2.74, which also had some anti-tumor effects. It could be found through pathological section that, 36 hrs after nanopaticle injection, most tumor cells were dead except only few survived at the tumor edge in HAS-NP+magnetic field group, and lamellar necrosis was also found in HAS-NP group. It was proved from histological point of view that ADM-GHMN had great anti-tumor function.

ADM-GHMN has the function of targeting initiative, and this magnetic chemotherapeutic nanoparticle has very good tumor-targeting and slow-release functions as well as the function of producing embolism in target tumor blood vessels. Experiments showed that, after given, ADM-GHMN were phagocytosed by endothelial cells, but penetrated into outer blood vessel diastem after 30 mins, and most were phagocytosed by tumor cells after 24 hours. Zhang Yangde et al injected 125I marked magnetic albumin nanoparticles though arteries and veins of rats with liver cancer, and the particles aggregated on the targeted location by the function of added magnetic field. The group having liver artery injection had highest radiation of liver cancer tissues and most obvious liver artery embolism level. In advanced experiments of magnetic albumin nanoparticle in liver cancer therapy, it was found that the group added by magnetic field had longer life span than the other groups, and pathological sections from three 60 days-survived rats showed that tumor tissues were substituted by fibrous and non-constructional tissues, so it had very fine anti-tumor effect.

ADM-GHMN has anti-tumor functions from at least 3 aspects: (1) with the magnetic field, aggregated nanoparticles produce embolism in tumor blood vessels causing lack of blood and oxygen in tumor in order to improve the sensitivity and effect of chemotherapeutics; (2) some of the non-aggregated nanoparticles enter into tumor tissue diastem through capillary endothelium cells, and then release the chemotherapeutics; (3) with the function of magnetic targeting and because of constructional differences between tumorous and normal tissues, nanoparticles selectively aggregate to tumorous tissues, therefore the level of chemotherapeutics has been improved. The therapeutics contained in nanoparticles has a slow-release process, which makes chemotherapeutics with high concentration last a longer time period and function differently at different stages of cell cycle in order to elevate anti-tumor effect. As chemotherapeutics only functions to tumor cells in multiplication cycle not to ones in G stage, and tumor cells are not in a synchronic cell cycle, chemotherapeutics with high concentration is contained in tumor tissues for a long time. In one word, ADM-GHMN could last life span and increase the survival rates of transplanted liver cancer animals, and it provides a new way of liver cancer therapy.

On one hand, in this research adriamycin was carried by nanoparticle for the purpose of slow release; while on the other hand, liver artery injection was used as a new way of administration in order to greatly decrease the deposition in heart and side effects of adriamycin, and it thus increased stay time in vivo and boosted therapeutic effect. It was reported in some other literatures that nanoparticle had property of anti-drug resistance. Multi-drug resistance (MDR) is one of the most key factors failing tumor chemotherapy, and naniparticle, a new administration system, is advantageous in MDR reversion. De Verdoere et al developed poly-cyano acrylic acid orth-forth lip nanoparticle, and its function of MDR reversion was proved through the research of its function to p388 cells. The advance research of this preparation will build a foundation of clinical use for this new liver cancer therapy with better liver targeting and less side effects.

### 4. Research in Rabbit In-Vivo Pharmacokinetics

4.1 Blood drug level—Time curve showed that the decrease slope of ADM-GHMN was greater than ADM; blood drug level of ADM-GHMN decreased faster than adriamycin; after 40′ blood drug level of ADM-GHMN maintained at a stable level 0.0778±0.0015 mg/l for a longer time while blood drug level of adriamycin was decreased (FIG. 6).

4.2 The pharmacokinetics rules of adriamycin and ADM-GHMN fit Three Compartment Model, and the weight was 1, AICs were −68.5984±16.7905 and −93.568±15.17, and fitting rates were 0.9923±0.0117 and 0.9936±0.005 (FIG. 7).

4.3 α of ADM-GHMN was 0.55 times of that of adriamycin, β was 0.2385 times of that of adriamycin, Vc was 1.0868 times of that of adriamycin, T1/2pi of both were similar, T1/2α was 3.2209 times of that of adriamycin, and T1/2β was 19152 times of that of adriamycin (FIGS. 7 and 8).

4.4 In Three Compartment Model the constant K12 of first-rate of transportation from central compartment to shallow peripheral compartment of ADM-GHMN was 2.4278 times of that of adriamycin, K21 of transportation from shallow peripheral compartment to central compartment was 0.1235 times of that of adriamycin, K13 from central compartment to deep peripheral compartment was 2.997 times of that of adriamycin, K31 of transportation from deep peripheral compartment to central compartment was 2.0077 times of that of adriamycin, and first elimination constant K10 of central compartment elimination was 0.4923 times of that of adriamycin.

4.5 Clearance rate of ADM-GHMN was 0.5368 times of that of adriamycin (FIG. 9).

4.6 Area under curve (AUC) of blood drug level—Time curve of ADM-GHMN was 1.3697 times of that of adriamycin (FIG. 10).

TABLE 3
pharmacokinetics parameterof adriamycin and ADM-GHMN

| pharmacokinetics | | |
|---|---|---|
| parameter | adriamycin | ADM-GHMN |
| P/mg · l | 0.0828 ± 0.0166 | 0.2137 ± 0.0365 |
| Gamma/m − 1 | 0.5463 ± 0.1663 | 0.5917 ± 0.0971 |
| A/mg · l − 1 | 0.1362 ± 0.0185 | 0.0020 ± 0.0033 |
| α/m − 1 | 0.0523 ± 0.0161 | 0.0288 ± 0.0662 |
| B/mg · l − 1 | 0.0109 ± 0.0028 | 0.0082 ± 0.001 |
| β/l · m − 1 | 0.0018 ± 0.0015 | 0.000421 ± 0.0002 |
| V c/(mg)/(mg/l) | 18.6816 ± 2.3663 | 20.3033 ± 3.8773 |
| T1/2pi/m − 1 | 1.3194 ± 0.3389 | 1.1827 ± 0.1762 |
| T1/2α/m | 13.8169 ± 3.5845 | 44.5027 ± 66.5753 |
| T1/2β/m | 883.2164 ± 2198.4914 | 1691.5525 ± 462.5832 |
| K12/l · m − 1 | 0.1651 ± 0.0668 | 0.4008 ± 0.3438 |
| K21/l · m − 1 | 0.3558 ± 0.1643 | 0.0440 ± 0.0627 |
| K13/l · m − 1 | 0.0513 ± 0.0188 | 0.1538 ± 0.2923 |
| K31/l · m − 1 | 0.0056 ± 0.0033 | 0.0112 ± 0.0068 |
| K10/l · m − 1 | 0.0227 ± 0.0171 | 0.011192 ± 0.0044 |
| AUC0→∞/m · mg/l | 14.8163 ± 24.789 | 20.2938 ± 4.3506 |
| C l/mg/m/(mg/l) | 0.4233 ± 0.3223 | 0.2273 ± 0.1111 |
| R2 | 0.9923 ± 0.0117 | 0.9936 ± 0.005 |
| AIC | −68.5984 ± 16.7905 | −93.568 ± 15.17 |

### BRIEF DESCRIPTION OF THE DRAWINGS

FIG. 1 Diameter of magnetic powder

FIG. 2 Magnetic powder under atomic force microscope

FIG. 3.1 Particle diameter

FIG. 3.2 Particle diameter

FIG. 3.3 Particle diameter

FIG. 4 Nano drug carrier under electron microscope

FIG. 5 Releasing of adriamycin and drug carrier nanoparticle

FIG. 6 Average blood drug level—Time Curve

FIG. 7 Distribution phase of biological half-life

FIG. 8 Elimination phase of biological half-life

FIG. 9 Elimination rate

FIG. 10 Blood drug level—Area under time curve

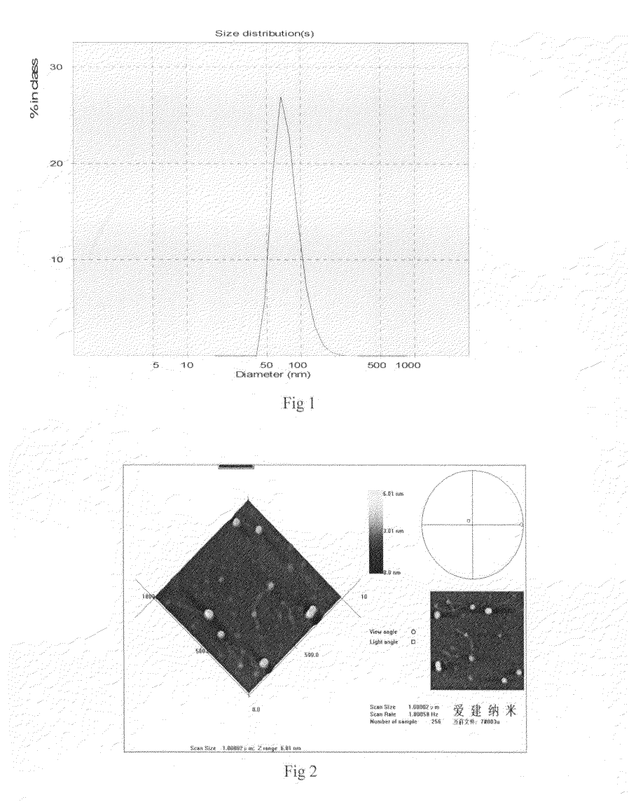

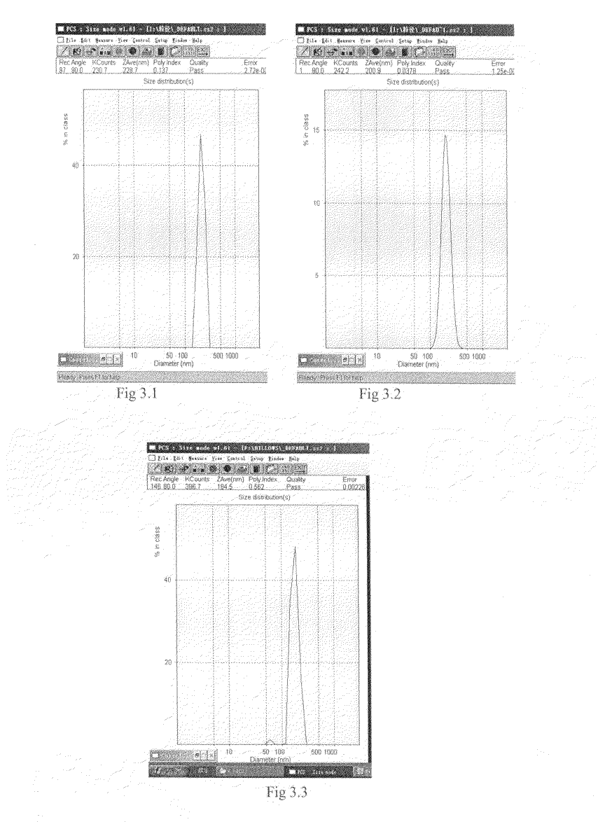

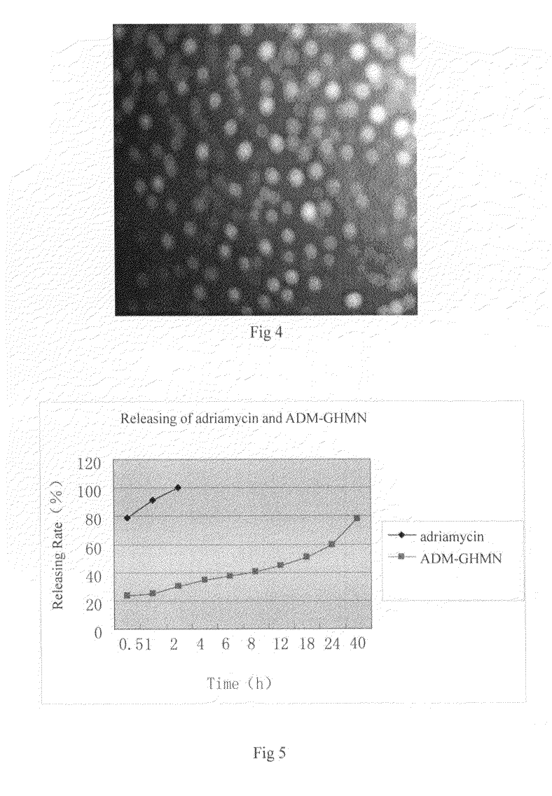

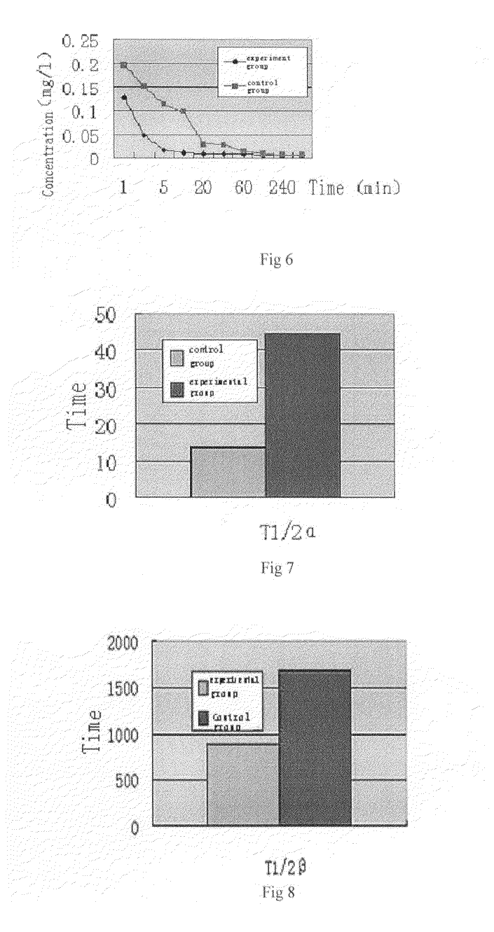

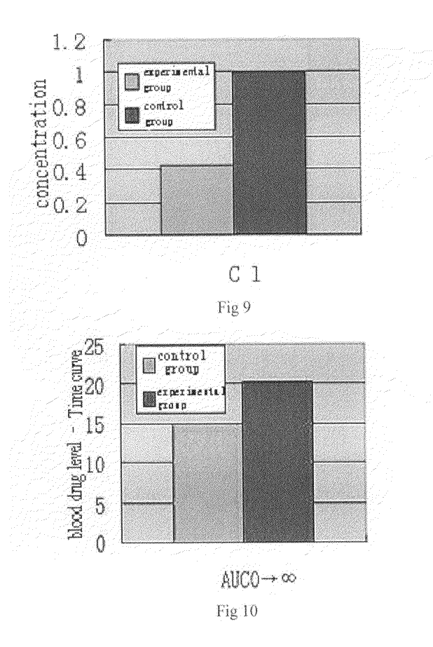

### DETAILED DESCRIPTION OF THE PREFERRED EMBODIMENTS

Practical approach is referenced to the above disclosed preparation and parameters of this application.

1. A method of making galactose albumin adriamycin magnetic nanoparticles, comprising:
(a) providing Fe₃O₄ magnetic-nanoparticles that have been treated with ultrasound;
(b) mixing said ultrasound treated Fe₃O₄ magnetic-nanoparticles of step (a) with galactosyl-HAS and adriamycin;
(c) providing a first portion of refined cottonseed oil to the mixture obtained in step (b)
(d) emulsifying the mixture obtained in step (c) by applying ultrasound to said mixture,
(e) adding dropwise the emulsified mixture obtained in step (d) to a second portion of heated refined cottonseed oil that is stirring;
(f) cooling the mixture obtained in step (e);
(g) adding ether to the cooled mixture obtained in step (f);
(h) rotating the mixture obtained in step (g) in a centrifuge;
(i) discarding the top layer of the mixture obtained in step (h) after centrifugation while retaining said galactose albumin adriamycin magnetic nanoparticles,
(j) washing said retained galactose albumin adriamycin magnetic nanoparticles; and
(k) drying said washed galactose albumin adriamycin magnetic nanoparticles.

2. The method of claim 1, wherein said step (d) is performed at 4° C.

3. The method of claim 1, wherein said step (e) is performed with refined cottonseed oil that is heated to 120° C.

4. The method of claim 2, wherein said step (e) is performed with refined cottonseed oil that is heated to 120° C.

5. The method of claim 1, wherein said step (e) is performed with refined cottonseed oil that is stirring at 2000 r/min.

6. The method of claim 2, wherein said step (e) is performed with refined cottonseed oil that is stirring at 2000 r/min.

7. The method of claim 3, wherein said step (e) is performed with refined cottonseed oil that is stirring at 2000 r/min.

8. The method of claim 4, wherein said step (e) is performed with refined cottonseed oil that is stirring at 2000 r/min.

9. The method of claim 1, wherein said step (h) is performed with a centrifuge rotating at 3000 r/min.

10. The method of claim 2, wherein said step (h) is performed with a centrifuge rotating at 3000 r/min.

11. The method of claim 3, wherein said step (h) is performed with a centrifuge rotating at 3000 r/min.

12. The method of claim 4, wherein said step (h) is performed with a centrifuge rotating at 3000 r/min.

13. The method of claim 5, wherein said step (h) is performed with a centrifuge rotating at 3000 r/min.

14. The method of claim 6, wherein said step (h) is performed with a centrifuge rotating at 3000 r/min.

15. The method of claim 7, wherein said step (h) is performed with a centrifuge rotating at 3000 r/min.

16. The method of claim 8, wherein said step (h) is performed with a centrifuge rotating at 3000 r/min.

17. A method of using a galactose albumin adriamycin magnetic nanoparticle to inhibit hepatic tumor cells comprising:
providing a galactose albumin adriamycin magnetic nanoparticle to a mammal having a hepatic tumor cell; and
determining the inhibition of said hepatic tumor cell in said mammal.

18. The method of claim 17, further comprising contacting said mammal with a magnetic field.

19. The method of claim 17, wherein the galactose albumin adriamycin magnetic nanoparticle obtained by the method of claim 1 is provided.

20. A slow release formulation of adriamycin comprising a galactose albumin conjugated magnetic nanoparticle joined to adriamycin.
